(19) United States
(12) Patent Application Publication
(10) Pub. No.: US 2017/0326318 A1
(43) Pub. Date: Nov. 16, 2017

(54) CRICOTHYROTOMY INTRODUCER TOOL

(71) Applicant: RICHARD VANNER, CHELTENHAM (GB)

(72) Inventor: RICHARD VANNER, CHELTENHAM (GB)

(21) Appl. No.: 15/592,707

(22) Filed: May 11, 2017

(30) Foreign Application Priority Data
May 13, 2016 (GB) 1608440.2

(51) Int. Cl. A61M 16/04

(52) U.S. Cl. CPC A61M 16/0472; A61M 16/0488

(57) ABSTRACT

A cricothyrotomy introducer tool comprising a guide member and a location member, wherein said guide member comprises a longitudinal axis and a guide surface for defining a path through the skin of a patient into the trachea, wherein said location member comprises a locating surface lateral to said longitudinal axis for contacting the skin of the patient to delimit the extent said guide member penetrates said trachea, and wherein said location member is movable between a first position wherein said locating surface is spaced from a first end of said guide member by a first distance, and a second position wherein said locating surface is spaced from said first end by a second distance.

### BACKGROUND

The present invention relates to a cricothyrotomy introducer tool for use particularly, but not exclusively, during an emergency cricothyrotomy procedure.

Emergency cricothyrotomy (sometimes called cricothyroidotomy) is a rarely performed lifesaving medical procedure undertaken when a patient ceases to breathe on their own, and the doctor cannot ventilate the lungs or intubate the trachea through the mouth. The procedure is usually performed by an anesthetist caring for a patient under general anesthetic during another surgical procedure. Cricothyrotomy involves making an incision on the front of the neck, and then passing a tube through the skin, subcutaneous tissue and the cricothyroid membrane into the trachea. The tube is then used to ventilate the lungs with oxygen.

The procedure is only performed about 58 times a year in the UK, which is only one in every 50,000 anesthetics. This equates to an average of less than once in the lifetime of an anesthetist. As such, the procedure is stressful for those who perform it, because it is difficult and unplanned, and because it is performed under time pressure when a patient is in critical need. As such, it has an unduly high failure rate. The procedure is not like a tracheotomy (or tracheostomy), which involves passing a tube below the cricoid cartilage directly into the trachea, and which is performed by a surgeon in a planned way.

One of the issues with cricothyrotomy is that the depth of subcutaneous tissue varies from person to person, and with an obese person it can be quite thick. This means it can be difficult to accurately locate the trachea, and successfully insert the tube. Sometimes harm is caused to the trachea by the incision, or by the insertion of the tube. Another issue is that the procedure involves a number of steps. It has recently been recommended that a procedure involving fewer steps would be easier.

### SUMMARY

The present invention is intended to address some of the above described problems. Therefore, according to the present invention a cricothyrotomy introducer tool comprises a guide member and a location member, in which said guide member comprises a longitudinal axis and a guide surface for defining a path through the skin of a patient into the trachea, in which said location member comprises a locating surface lateral to said longitudinal axis for contacting the skin of the patient to delimit the extent said guide member penetrates said trachea, and in which said location member is movable between a first position in which said locating surface is spaced from a first end of said guide member by a first distance, and a second position in which said locating surface is spaced from said first end by a second distance.

Thus, the present invention provides a tool specifically adapted for performing cricothyrotomy, and it addresses two of the main problems associated with the conventional approach. Firstly, the tool comprises the guide surface, which is used to direct the tube into the trachea. The operator can apply the tube to the surface and drive it along the surface into the correct position. Secondly, the tool comprises the movable locating surface to ensure that the guide surface is located at the correct depth in the patient's neck to guide the tube into position. In particular, the locating surface can be positioned for two or more kinds of patient. As described further below, at the very least this can be for an average adult female and an average adult male, but it can also include positions for larger and obese patients. The user merely needs to set the tool according the patient's size, and then proceed.

The guide surface can be used to guide two different kinds of tube into the trachea. Firstly, the tube could be one which is intended to provide a passageway to ventilate the lungs of the patient. Such tubes are known, and are made of a resilient material which can flex as it is driven down the guide surface into the trachea by the user. However, more beneficially the tube can actually be a means to locate a more suitable passageway to ventilate the lungs. In particular, the tube can be a bougie, which is a known kind of single use resilient rod which is commonly passed into the trachea through the mouth. A cuffed ended-tracheal tube (CETT) can be mounted to the bougie, and can be railroaded into the trachea over the bougie. This procedure is explained more fully below. As an alternative a fibre-optic bronchoscope could be used in place of a bougie.

It will be appreciated that the location member can be any mechanical device comprising a laterally extending part to provide for the locating surface. This could just be a flange of any shape which is axially movable on the guide member. However, in a preferred construction the location member can comprise a tube portion, and the guide member can comprise a rod portion located inside the tube portion. The tube portion and rod portion can be co-axial on the longitudinal axis, and can be axially moveable in relation to one another. With this construction a first end of the tube portion can comprise the locating surface. This is an ergonomic and efficient configuration, because the tube portion of the locating member can be used as an operating handle.

Preferably the tool can comprise a releasable locking mechanism which can axially lock the tube portion and the rod portion together in the first position and the second position. In the first position and the second position the guide member can protrude from the first end of the tube portion by the first distance and the second distance respectively.

It will be appreciated that the tube portion can just be a regular tube along its whole longitudinal length, however this would mean that the locating surface would be wholly annular as well as perpendicular to the longitudinal axis of the guide member, which is not an ergonomic shape which particularly matches the neck of the patient. Therefore, in a preferred construction the tube portion can comprise a splayed section which increases in diameter from a mid-point of the tube portion to the first end thereof, and the splayed section can further comprise a longitudinal curvature such that the first end of the tube portion is laterally displaced from the longitudinal axis of the guide member. With these features the locating surface has a larger diameter than the tube portion, which makes it more effective at fulfilling its purpose. The longitudinal curvature allows for a particularly beneficial shape of locating surface to be formed on the outside circumference of the curve, which is explained further below.

The splayed section can comprise a first axially extending notch, and the rod portion can comprise a trough section which extends from a mid-point of the rod portion to the first end of the guide member, an inner surface of which can form the guide surface. The rod portion can be rotationally arranged inside the tube portion such that the inner surface of the trough section can radially face the first notch in the first position and the second position. Therefore, when the tool is correctly located in the patient and the locating surface is in contact with their neck, the user can pass the tube through the first notch and onto the guide surface. It can then be driven down the guide surface into the trachea.

Furthermore, the splayed section can comprise a second axially extending notch which can be radially opposite to the first notch, and the second notch can be at an oblique angle to the longitudinal axis of the guide member. Along with the longitudinal curvature of the splayed section this feature creates a more complex shape of locating surface which is optimal for the operation of the tool. In particular, the locating surface can comprise tips of the first end of the tube portion between the first notch and the second notch, and a leading edge of the second notch. Therefore, when the tool is first inserted into the patient this is done until the tips described below first contact the skin. The tool is then rotated in the sagittal plane until the leading edge of the second notch contacts the skin. With this configuration the locating surface not only determines the depth of penetration of the tool into the patient, but it also assists the user in correctly positioning the tool in the sagittal plane so the guide surface leads into the middle of the trachea. It will be appreciated that as the splayed section comprises the longitudinal curvature, so does the leading edge of the second notch. This provides an ergonomic shape which is suitable for application to the relevant part of the patient's neck.

The trough section can also comprise a longitudinal curvature such that the first end of the guide member is radially displaced from the longitudinal axis. This feature ensures that the guide surface directs the tube from the angle of the tool at the point of incision to an angle which is substantially aligned with the coronal plane. This can prevent the tube from contacting the rear part of the patient's trachea when it enters, and aligns the tube with the direction of the trachea.

It will be appreciated that the cricothyrotomy tool of the invention can be inserted into a patient after an incision has been made in the neck. However, preferably the tool can also be used to make the incision itself. Therefore, in a preferred embodiment the first end of the guide member can be flattened to a cutting blade. Therefore, the user can use the tool to create the incision and position the guide surface in one step.

As referred to above, the rod portion can be rotationally arranged inside the tube portion such that the inner section of the trough section can radially face the first notch. This is obviously important to ensure that the guide surface is immediately accessible to the user once the tool has been inserted into the patient. It will be appreciated that the tube portion and the rod portion can be maintained in this fixed rotational arrangement by means of any known mechanical arrangement. For example, an axially extending trough can be formed in the rod portion, which can engage an axially extending rail formed on the inside surface of the tube portion. However, preferably a second end of the tube portion can comprise a third axially extending notch, and the rod portion can comprise a radially extending boss passing through the third notch. The boss moves up and down the third notch when the rod portion moves axially in relation to the tube portion, and in all axial positions of the rod portion it prevents it rotating in relation to the tube portion.

It will be appreciated that the first distance and the second distance can be determined by the skilled person according to their own research and requirements. However, it has been found that for an average female patient the distance between the point of incision and the middle of the trachea is about 20 mm, and for an average male patient the distance is about 25 mm. Therefore, preferably in the first position the guide member can protrude from the first end of the tube portion by substantially 20 mm, and in the second position it can protrude by substantially 25 mm.

As referred to above, given that the rod portion and the tube portion can be axially arranged in relation to one another in any position, the tool can accommodate more than just two use positions. Therefore, preferably the location member can be movable to three further positions in which the locating surface is spaced from the first end of the guide member by three further distances. In these three further positions the guide member can protrude from the first end of the tube portion by substantially 30 mm, 35 mm and 40 mm respectively. These positions are suitable for larger or obese patients, and the user can adopt them according to their own judgement.

It will be appreciated that the tool could be used for even larger patients, or for smaller patients such as children, if it is provided with suitable dimensions, and the invention includes any such sizes of tool.

The tool is preferably made from metal parts, but it can also be made from other suitable materials, such as any known plastics material. A combination of materials is also possible, for example the guide member can be made from metal, and the location member can be made from a hard plastics material.

It will be appreciated that the locking mechanism can be any known mechanical mechanism for axially locking a rod inside a tube. However, in a preferred construction the locking mechanism can comprise a number of radially facing apertures provided in the rod portion, and a pin provided on the location member which is removably locatable in each of the radially facing apertures to axially lock the tube portion and the rod portion together.

The location member can then comprise a knob which can be manually operable to remove the pin from the apertures. The knob can be spring loaded against removal of the pin from said apertures, in order to ensure that the rod portion does not come lose in use.

Preferably the guide member can also be moved to a stowed position in which the first end of the guide member is positioned rearwardly of the locating surface. With this feature the cutting blade formed at the first end of the guide member can be retracted into the tool to protect the user, and to make the tool more readily portable without the need for bulky protective packaging. If a locking mechanism like that described above is used, then this feature can be facilitated by the pin being manually removed from the apertures and the guide member then being manually moved into the stowed position, perhaps by manipulation of the boss up towards the top of the third notch. The pin can then just rest against the guide member, or alternatively an additional aperture can be provided to lock the guide member in the stowed position. In one embodiment the pin can protrude into the trough section to lock the rod portion in the stowed position.

The invention can be performed in various ways, but one embodiment will now be described by way of example, and with reference to the accompanying drawings.

### BRIEF DESCRIPTION OF THE DRAWINGS

FIG. 1 is a side view of a cricothyrotomy introducer tool according to the present invention;

FIG. 2 is a plan view of the cricothyrotomy introducer tool as shown in FIG. 1;

FIG. 3 is a cross-sectional side view of the cricothyrotomy introducer tool as shown in FIG. 1 in a first configuration;

FIG. 4 is a cross-sectional side view of the cricothyrotomy introducer tool as shown in FIG. 1 in a second configuration;

FIG. 5 is a cross-sectional side view of the cricothyrotomy introducer tool as shown in FIG. 1 in a third configuration;

FIG. 6 is a cross-sectional side view of the cricothyrotomy introducer tool as shown in FIG. 1 in a stowed position;

FIG. 7 is a diagrammatic view of the cricothyrotomy introducer tool as shown in FIG. 1 in the first position of a first step of a cricothyrotomy procedure;

FIG. 8 is a diagrammatic view of the cricothyrotomy introducer tool as shown in FIG. 1 in the second position of a first step of a cricothyrotomy procedure;

FIG. 9 is a diagrammatic view of the cricothyrotomy introducer tool as shown in FIG. 1 during a second step of a cricothyrotomy procedure;

FIG. 10 is a diagrammatic view of the third step of a cricothyrotomy procedure;

FIG. 11 is a diagrammatic view of the fourth step of a cricothyrotomy procedure; and

FIG. 12 is a diagrammatic view of the fifth step of a cricothyrotomy procedure.

### DETAILED DESCRIPTION

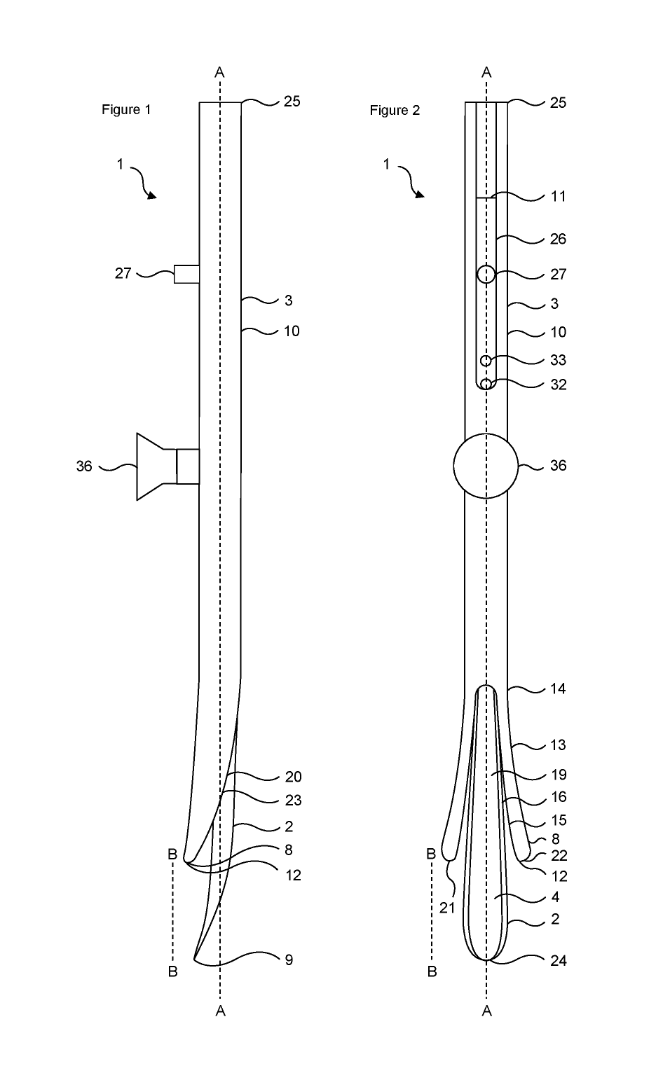

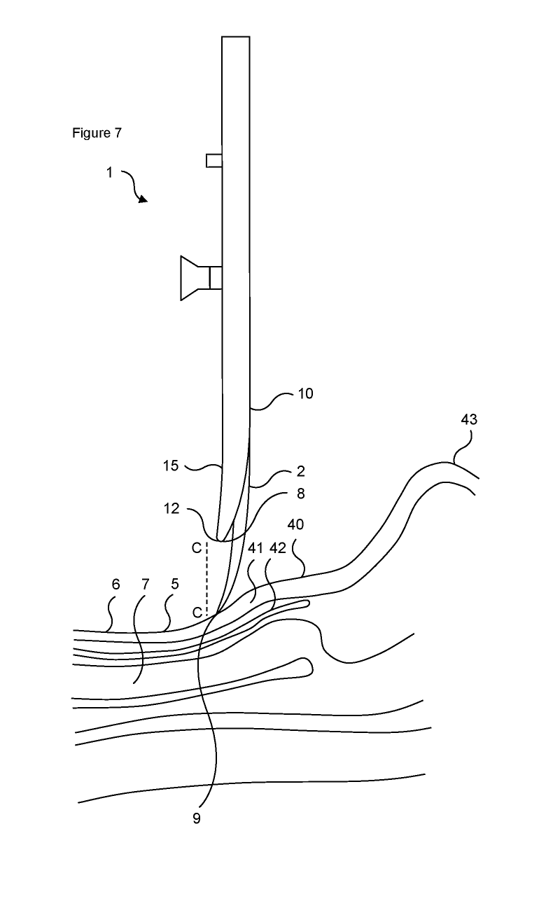

As shown in the Figures a cricothyrotomy introducer tool 1 comprises a guide member 2 and a location member 3. The guide member 2 comprises a longitudinal axis A-A and a guide surface 4 for defining a path through the skin 5 of a patient 6 into the trachea 7. The location member 3 comprises a locating surface 8 lateral to the longitudinal axis A-A for contacting the skin 5 of the patient 6 to delimit the extent the guide member 2 penetrates the trachea 7. As explained further below, the location member 2 is movable between a first position, as shown in FIGS. 1-4 in which the locating surface 8 is spaced from a first end 9 of the guide member 2 by a first distance B-B, and a second position, as shown in FIGS. 7-9 in which the locating surface 8 is spaced from the first end 9 by a second distance C-C.

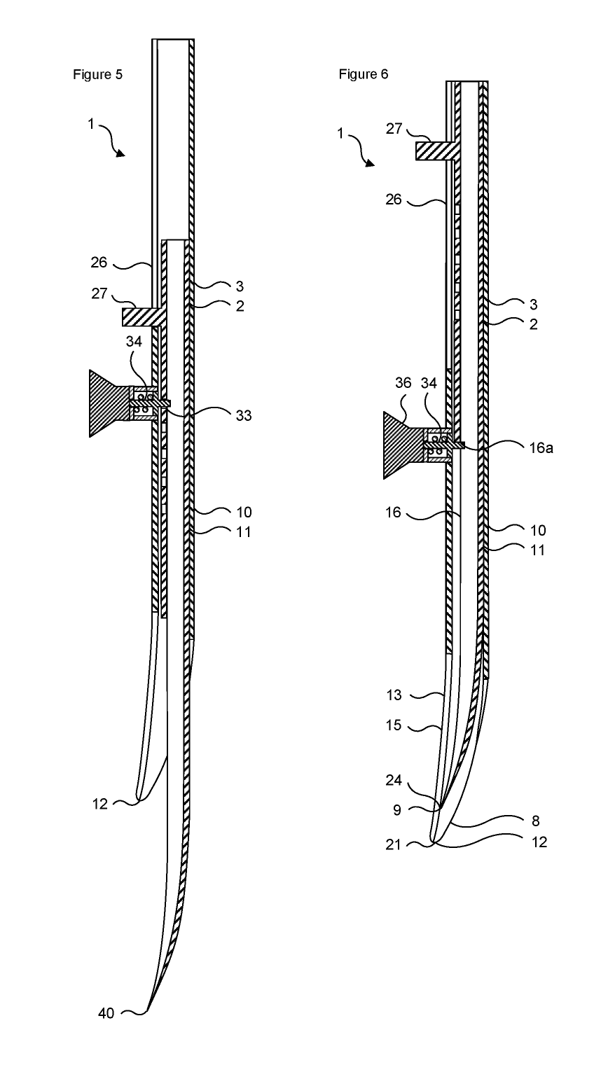

As is clear from the Figures, the location member 3 comprises a tube portion 10, and the guide member 2 comprises a rod portion 11 located inside the tube portion 10. The tube portion 10 and rod portion 11 are co-axial on the longitudinal axis A-A, and are axially moveable in relation to one another, as illustrated by the difference between FIGS. 4 and 5. The tube portion 10 serves as an operating handle for the tool 1, as it can be readily manipulated by the user. A first end 12 of the tube portion 10 comprises the locating surface 8, which the user can apply to the skin of the patient by manually manipulating the tube portion 10.

The tube portion 10 is 170 mm long and has an outside diameter of 12 mm. The rod portion 11 is about 160 mm long and has an outside diameter of 8 mm. Both the tube portion 10 and the rod portion 11 are made from metal.

The tube portion 10 comprise a splayed section 13 which increases in diameter from a mid-point 14 of the tube portion 10 to the first end 12 thereof. Further, as is shown in the Figures the splayed section 13 comprise a longitudinal curvature, such that the first end 12 of the tube portion 10 is laterally displaced from the longitudinal axis A-A of the guide member 2. The splayed section 13 also comprises a first axially extending notch 15. As shown in FIG. 2 this is generally U shaped.

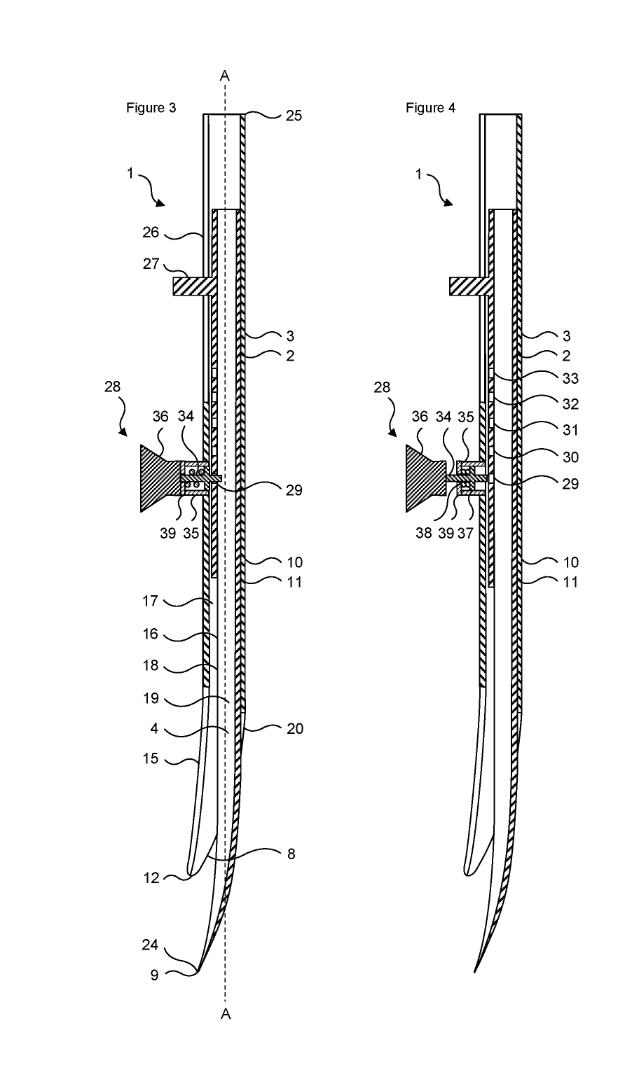

Referring to FIG. 3, the rod portion 11 comprises a trough section 16 which extends from a mid-point 17 of the rod portion 11 to the first end 9 of the guide member 2. The trough section 16 is 75 mm in length. As is clear from the Figures the rod portion 11 is a hollow tube, and the trough section 16 is formed by an axially extending cut-away section 18 which leaves only a part of the tube remaining. An inner surface 19 of the trough section 16 has a diameter of 5.5 mm and comprises the guide surface 4. As is clear from FIGS. 2 to 6, the rod portion 11 is rotationally arranged inside the tube portion 10 such that the inner surface 19 of the trough section 16 radially faces the first notch 15. As explained further below, this allows the bougie 46 to be introduced to the guide surface 4 through the first notch 15 when the tool 1 is inserted into the neck of the patient 6.

The splayed section 13 comprises a second axially extending notch 20 which is radially opposite to the first notch 15. As is clear from FIGS. 1 and 3, the second notch 20 is arranged at an oblique angle to the longitudinal axis A-A of the guide member.

As a result of these features a complex shape of locating surface 8 is formed. In particular, referring to FIGS. 1 and 2 the locating surface 8 comprises the remaining tips 21 and 22 of the first end 12 of the tube portion 10 between the first notch 15 and the second notch 20, and a leading edge 23 of the second notch 20. As the splayed section 13 comprises a curvature, so does the leading edge 23 of the second notch 20. As described further below, this provides an ergonomic shape which is suitable for application to the relevant part of the patient's neck.

The trough section 16 also comprises a longitudinal curvature such that the first end 9 of the guide member 2 is laterally displaced from the longitudinal axis A-A. Furthermore, the first end 9 of the guide member 2 is flattened to a cutting blade 24.

The flattened section is about 10 mm in length, and the cutting blade 24 is about 10 mm wide. It makes a horizontal incision in the skin and tissues, which can be filed by the trough section 16 as the guide member 2 is moved into position, as explained further below.

A second end 25 of the tube portion 10 comprises a third axially extending notch 26, and the rod portion 11 comprises a radially extending boss 27 passing through the third notch 26. The boss 27 moves up and down the third notch 26 when the rod portion 11 moves axially in relation to the tube portion 10, and in all axial positions of the rod portion 11 it prevents it rotating in relation to the tube portion 10. This ensures that a user gripping the tube portion 10 as a handle has control over the cutting blade 24 and the position of the guide member 2 generally. It also ensures that the trough section 16 is properly aligned with the first notch 15.

Referring to FIG. 4, the tool 1 comprises a releasable locking mechanism, generally designated 28, which allows for the guide member 2 to be locked in five axial use positions relative to the location member 3, and in a stowed position. In particular, the rod portion 11 comprises five radially facing apertures 29-33, and a pin 34 is provided on the location member 3 which is removably locatable in each of the radially facing apertures 29-33 to axially lock the tube portion 10 and the rod portion 11 together.

The locking mechanism 28 comprises a housing 35 provided on the tube portion 10, in which the pin 34 is mounted. The pin 34 comprises an operation knob 36, and it has a flange 37 disposed inside the housing 35. A spring 38 is located between the flange 37 and a top 39 of the housing 35, which biases the pin 34 into the apertures 29-33. In FIG. 3 the pin 34 is biased into the first aperture 29. This biasing is delimited by the knob 36 abutting against the top 39 of the housing 35. The knob 36 can be manually lifted against the force of the spring 38 to withdraw the pin 34 from any of the apertures 29-33, as shown in FIG. 4. Therefore, to adjust the axial position of the rod portion 11 in relation to the tube portion 10 the knob 36 is lifted and the rod portion 11 manipulated into a different axial position. This is most readily achieved by moving the boss 27 up and down the third notch 26. When the desired position is reached the knob 36 is released and the force of the spring 38 moves the pin 34 into the aperture 29-33 below, thereby locking the rod portion 11 in place.

The apertures 29-33 are spaced apart from each other by 5 mm. When the pin 34 is located in the first aperture 29 the guide member 2 protrudes from the first end 12 of the tube portion 10 by 20 mm. This is the first distance B-B shown in FIGS. 1 and 2. This distance is suitable for an average adult female, as it is the distance between the front of their neck and the middle of the trachea. As such, when the locating surface 8 contacts their skin the first end 9 of the guide member 2 will be located in the middle of the trachea.

When the pin 34 is located in the second aperture 30 the guide member 2 protrudes from the first end 12 of the tube portion 10 by 25 mm. This is the second distance C-C shown in FIGS. 7-9. This distance is suitable for an average adult male, as it is the distance between the front of their neck and the middle of the trachea. As such, when the locating surface 8 contacts their skin the first end 9 of the guide member 2 will be located in the middle of the trachea 7.

When the pin 34 is located in the third, fourth and fifth apertures 31-33 the guide member 2 protrudes from the first end of the tube portion 10 by 30 mm, 35 mm and 40 mm respectively. These distances are suitable for larger or obese patients, as these can be the distances between the front of their neck and the middle of the trachea. With obese patients an ultrasound machine can be used to assess the depth from the skin to the middle of the trachea, as well as the position of the cricothyroid membrane, before induction of anesthesia, so the user does not have to guess at the depth if the tool 1 has to be used. In FIG. 5 the pin 34 is located in the fifth aperture 33, and the guide member 2 protrudes from the first end 12 of the tube portion 10 by 35 mm. In this position the boss 27 abuts up against the end of the third notch 26, and as such the guide member 2 cannot protrude from the first end 12 of the tube portion 10 any further than 35 mm.

The guide member 2 can also be moved to a stowed position, as shown in FIG. 6, in which the first end 9 is positioned rewardly of the locating surface 8, or at least rearwardly of the front most part thereof at the tips 21 and 22. To achieve this position the knob 36 is lifted so the pin 34 is removed from the apertures 29 to 33, and the rod portion 11 manipulated into the most rearward position possible. This is most readily achieved by moving the boss 27 up towards the top of the third notch 26. The knob 36 is released and the pin 34 protrudes into the trough section 16, which locks the rod portion 11 in place in the stowed position. An inner end 16a of the trough section abuts against the pin 34, so the rod portion 11 is prevented from moving forward. In this position first end 9 of the rod portion 11 is housed within the splayed section 13, and the cutting blade 24 does not protrude from the first end 12 of the tube member 10, nor laterally outward through the first notch 15. As such, the tool 1 can be more safely handled by the user, and is more readily portable without the need for protective packaging.

In use the tool 1 operates as follows. Firstly the user adjusts the tool 1 so the guide member 2 protrudes from the first end 12 of the tube portion 10 by the correct distance for the patient 6. The tool 1 should be provided initially in the stowed position as shown in FIG. 6. In order to set it for use, the knob 36 is manually lifted against the force of the spring 38 to release the rod portion 11 from the stowed position, and the rod portion 11 is then manually moved to the desired position by moving the boss 27 down the third notch 26. When the rod portion 11 is in the desired position the knob 36 is released and the pin 34 enter the aperture 29-33 below it to lock it in place. If the patient is an adult female of average size the guide member 2 is positioned so it protrudes from the first end 12 of the tube portion 10 by 20 mm, and the pin 34 is placed in the first aperture 29, as shown in FIG. 3. If the patient is an adult male of average size the guide member 2 is positioned so it protrudes from the first end 12 of the tube portion 10 by 25 mm, and the pin 34 is placed in the second aperture 30. This is the configuration shown in FIGS. 7-9. If the patient is larger or obese the guide member 2 is positioned so it protrudes from the first end 12 of the tube portion 10 by either 30 mm, 35 mm or 40 mm according to the requirement, and the pin 34 is placed in the third 31, fourth 32 or fifth aperture 33 as required. FIG. 5 shows the guide member 2 at its maximum extension, with the pin 34 placed in the fifth aperture 33.

FIGS. 7-12 are diagrammatic views of the steps of the cricothyrotomy procedure performed on an adult male 6 of average size, whose neck region 40 only is shown. He is of normal anatomy and has skin 5, subcutaneous tissue 41, cricothyroid membrane 42 and trachea 7.

In order to prepare the patient 6 for the cricothyrotomy procedure their pillow is removed, and the head 43 extended and skin 5 cleaned if time. A right-handed operator should stand on the patient's left side and vice versa.

In the first step of the cricothyrotomy procedure the tool 1 is inserted into the patient's neck 40. The skin 5 over the cricothyroid membrane 42 is located and slightly stretched by the forefinger and thumb of the user's non-dominant hand. An initial 9 mm skin incision with a size 20 scalpel blade can be made to assist the entry of the tool 1. The tool 1 is held with the dominant hand by the tube portion 10, in the patient's mid line with the first notch 15 facing caudally, and the tube portion 10 vertical so it is at 90 degrees to the skin 5. This position is shown in FIG. 7. The tool 1 is then advanced through the skin 5 and cricothyroid membrane 42. The cutting blade 24 cuts through the subcutaneous tissue 41 and the cricothyroid membrane 42, creating a path for the trough section 16 to follow. The operator will feel a slight give as the first end 9 of the guide member 2 enters the trachea 7.

The tool 1 is then advanced into the trachea 7 by about 7 mm or so, or until the locating surface 8 touches the skin 5. Initially it is the tips 21 and 22 of the first end 12 of the tube portion 10 which contact the skin 5, as the tool 1 is arranged vertically.

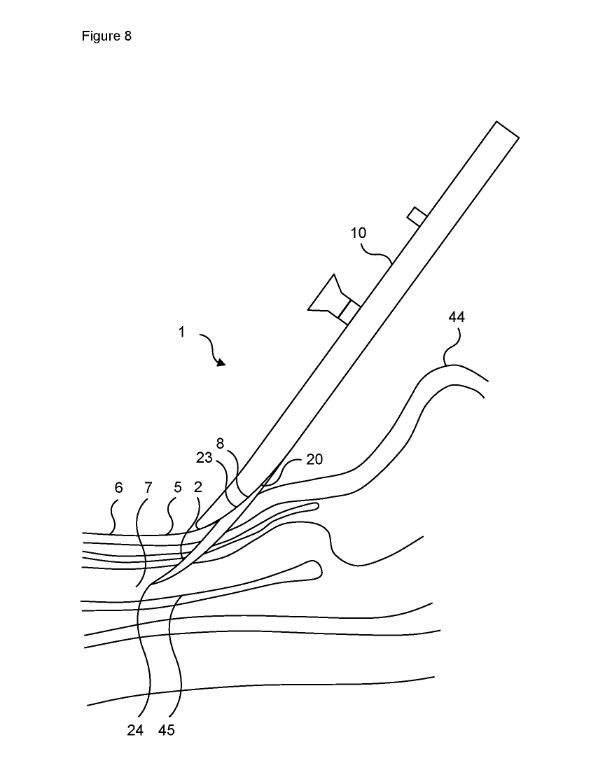

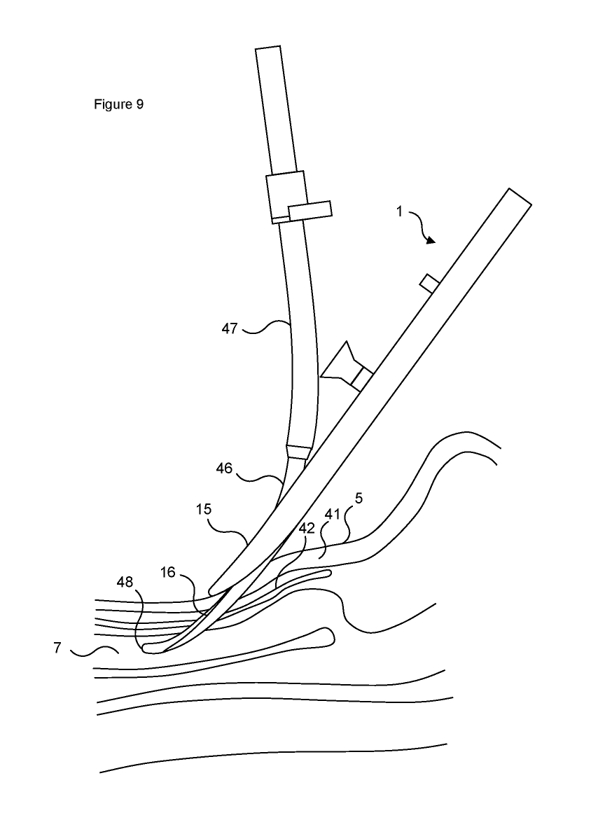

The tube portion 10 is then rotated in the sagittal plane from 90 degrees to approximately 45 degrees by lowering the tube portion 10 towards the chin 44. The leading edge 23 of the second notch 20 then contacts the skin 5. This is the position shown in FIG. 8, and it can be seen from this Figure that the particular angled and curved shape of the leading edge 23 of the second notch 20 is ergonomic and suitable for interface with the anatomy of the patient 6. In addition, the particular angle of the second notch 20 assists the user in achieving the correct 45 degree angle required, because as shown in FIG. 8 it contacts the skin 5 of the patient when the tool 1 reaches this angle. As is also clear from FIG. 8, the depth the guide member 2 is limited to by the locating surface 8, as well as the anterior curved shape of both the tube portion 10 and the guide member 2, ensure that the sharp cutting blade 24 passes down into the trachea 7 and avoids the posterior wall 45 thereof. This reduces the liklihood of damage to posterior structures.

In the second step of the cricothyrotomy procedure the user uses the their dominant hand to pass a bougie 46 about 80 mm through the tool 1 and into the trachea 7. The bougie 46 is a 14 FG size for a 5.0 mm internal diameter (ID) CETT 47, but it could also be a 15 FG size for a 6.0 mm ID CETT. The tip 48 of the bougie 46 is first passed through the first notch 15 and into the trough section 16, where it contacts the guide surface 4. The bougie 46 is then driven by the user down the guide surface 4 inside the trough section 16, and it travels through the skin 5, subcutaneous tissue 41 and cricothyroid membrane 42 and into the trachea 7. The shape of the guide surface 4 ensures that the resilient bougie 46 is biased from the angle of the tool 1 at the point of incision to an angle which is substantially aligned with the coronal plane. This is the position shown in FIG. 9.

It will be appreciated that this step relies on the rod portion 11 being rotationally locked inside the tube portion 10, so the guide surface 4 is immediately accessible to the user through the first notch 15. This is facilitated by the boss 27 being located in the third notch 26, so the rod portion 11 is prevented from rotating.

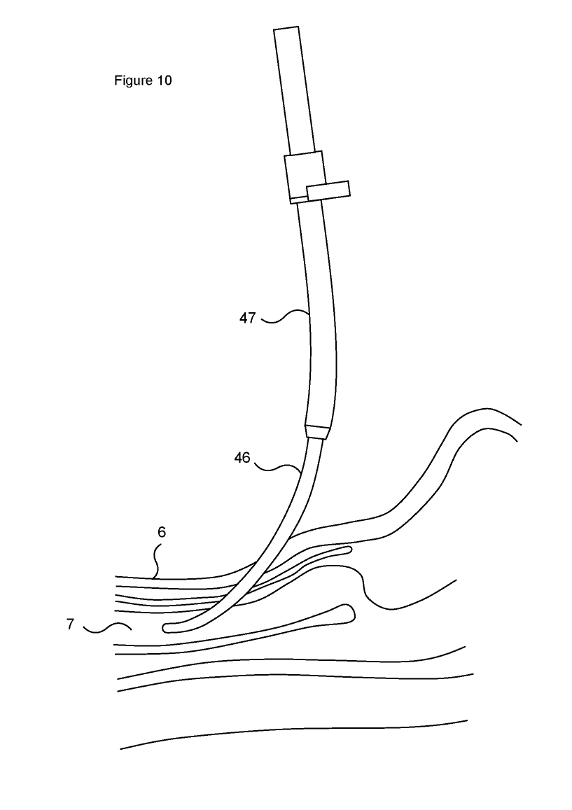

In the third step of the cricothyrotomy procedure the tool 1 is removed from the patient 6, leaving the bougie 46 and its associated CETT 47 in place. The bougie 46 must be held in place by the user while the tool 1 is removed, to keep it in place. FIG. 10 shows the position once the tool 1 has been removed. At this stage the tool 1 has performed its purpose, as the bougie 46 is in the correct position, in the middle of the trachea 7. The tool 1 has cut a large enough channel through the skin 5, subcutaneous tissue 41 and cricothyroid membrane 42 so the CETT 47 can pass more easily over the bougie 46.

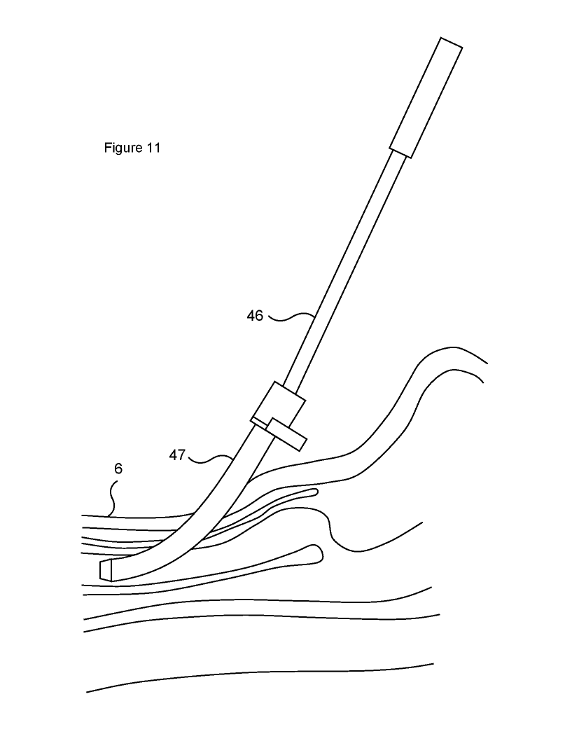

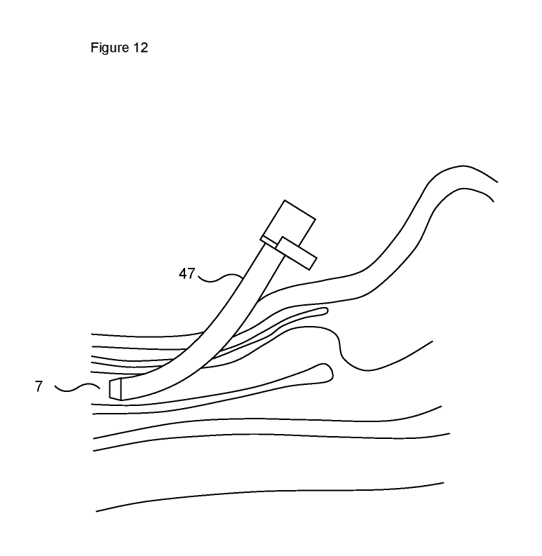

In the fourth step of the cricothyrotomy procedure the CETT 47 is pushed down the bougie 46 until it is in position in the patient 6. The user should use their dominant hand to perform this movement, while using their other hand to hold the cricoid cartilage of the larynx. FIG. 11 shows the position once this movement has been performed. In the final step the bougie 46 is removed, leaving the CETT 47 in place. This is the position shown in FIG. 12. The CETT 47 can then be used in the known way to ventilate the lungs of the patient 6 with oxygen. A capnograph trace can confirm that the CETT 47 is in the correct position in the trachea 7.

The present invention can be performed in other ways without departing from the scope of claim 1. For example, in one alternative embodiment (not shown) the location member is a flange which is axially movable on the guide member as opposed to being a tube arranged around the guide member. In another alternative embodiment (not shown), in order to rotationally lock the rod portion and the tube portion together an axially extending trough is formed in the rod portion, which engages an axially extending rail formed on the inside surface of the tube portion.

Therefore, the present invention provides a tool which addresses two of the main problems associated with the known cricothyrotomy method. Firstly, the guide surface directs the bougie into the correct position, and secondly the locating surface ensures that the guide surface is located at the correct depth in the patient's neck to guide the bougie into position. Of particular benefit is the adjustability of the position of the locating surface, so the tool can be used for many different sizes of patient. The most useful being when it is set for use with an obese patient, whose trachea would otherwise be difficult to manually locate.

1. A cricothyrotomy introducer tool comprising a guide member and a location member, in which:
said guide member comprises a longitudinal axis and a guide surface for defining a path through the skin of a patient into the trachea;
said location member comprises a locating surface lateral to said longitudinal axis for contacting the skin of the patient to delimit the extent said guide member penetrates said trachea; and
said location member is movable between a first position in which said locating surface is spaced from a first end of said guide member by a first distance, and a second position in which said locating surface is spaced from said first end by a second distance.

2. A cricothyrotomy introducer tool as claimed in claim 1 wherein:
said location member comprises a tube portion;
said guide member comprises a rod portion located inside said tube portion,
said tube portion and said rod portion are co-axial on said longitudinal axis, and are axially moveable in relation to one another; and
a first end of said tube portion comprises said locating surface.

3. A cricothyrotomy introducer tool as claimed in claim 2 further comprising:
a releasable locking mechanism which axially locks said tube portion and said rod portion together in said first position and said second position; and
in said first position and said second position said guide member protrudes from said first end of said tube portion by said first distance and said second distance respectively.

4. A cricothyrotomy introducer tool as claimed in claim 3 wherein said tube portion comprises:
a splayed section which increases in diameter from a mid-point of said tube portion to said first end thereof;
said splayed section further comprising a longitudinal curvature such that said first end of said tube portion is laterally displaced from said longitudinal axis.

5. A cricothyrotomy introducer tool as claimed in claim 4 wherein said splayed section comprises:
a first axially extending notch, wherein said rod portion comprises trough section which extends from a mid-point of said rod portion to said first end of said guide member;
an inner surface of which forms said guide surface; and
wherein said rod portion rotationally arranged inside said tube portion such that said inner surface of said trough section radially faces said first notch in said first position and said second position.

6. A cricothyrotomy introducer tool as claimed in claim 5 wherein said splayed section comprises:
a second axially extending notch which is radially opposite to said first notch; and
wherein said second notch is at an oblique angle to said longitudinal axis.

7. A cricothyrotomy introducer tool as claimed in claim 6 wherein said locating surface comprises:
tips of said first end of said tube portion between said first notch and said second notch; and
a leading edge of said second notch.

8. A cricothyrotomy introducer tool as claimed in claim 7 wherein said trough section comprises a longitudinal curvature such that said first end of said guide member is radially displaced from said longitudinal axis.

9. A cricothyrotomy introducer tool as claimed in claim 8 wherein said first end of said guide member is flattened to a cutting blade.

10. A cricothyrotomy introducer tool as claimed in claim 9 wherein:
a second end of said tube portion comprises a third axially extending notch; and
said rod portion comprises a radially extending boss passing through said third notch.

11. A cricothyrotomy introducer tool as claimed in claim 3 wherein in said first position said guide member protrudes from said first end of said tube portion by substantially 20 mm.

12. A cricothyrotomy introducer tool as claimed in claim 11 wherein in said second position said guide member protrudes from said first end of said tube portion by substantially 25 mm.

13. A cricothyrotomy introducer tool as claimed in claim 12 wherein:
said location member is movable to three further positions;
said locating surface is spaced from said first end of said guide member by three further distances; and
in said three further positions, said guide member protrudes from said first end of said tube portion by substantially 30 mm, 35 mm and 40 mm respectively.

14. A cricothyrotomy introducer tool as claimed in claim 3 wherein said locking mechanism comprises:
a number of radially facing apertures provided in said rod portion; and
a pin provided on said location member which is removably locatable in each of said radially facing apertures to axially lock said tube portion and said rod portion together.

15. A cricothyrotomy introducer tool as claimed in claim 14 wherein said location member comprises:
a knob which is manually operable to remove said pin from said apertures; and
wherein said knob is spring loaded against removal of said pin from said apertures.
